(19) United States
(12) Patent Application Publication
(10) Pub. No.: US 2010/0273787 A1
(43) Pub. Date: Oct. 28, 2010

(54) KYNURENINE-AMINOTRANSFERASE INHIBITORS

(76) Inventors: Robert Schwarcz, Baltimore, MD (US); Yasushi Kajii, Osaka (JP); Shin-Ichiro Ono, Osaka (JP)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/742,171

(22) PCT Filed: Nov. 13, 2008
(86) PCT No.: PCT/US08/83321

§ 371 Date: May 10, 2010

Related U.S. Application Data
(60) Provisional application No. 60/988,231, filed on Nov. 15, 2007.

(51) Int. Cl. A61K 31/5383; C07D 498/06; A61P 25/28; A61P 25/16; A61P 25/18; A61P 25/24; A61P 25/30; A61P 33/06

(52) U.S. Cl. 514/230.2; 544/101

(57) ABSTRACT

Compounds of formula (I): prodrug derivatives and/or pharmaceutically acceptable salt thereof, selectively inhibit the enzyme kynurenine aminotransferase, thereby reducing the synthesis of kynurenic acid. The compounds are used for the treatment of psychiatric and neurological diseases which benefit from an increase in glutamatergic and/or cholinergic neurotransmission, such as schizophrenia, depression, bipolar illness, anxiety and Alzheimer's disease. Furthermore, the compounds of the invention are useful for stimulating attention, memory and other cognitive processes in normal individuals of any age, including children, adolescents and the elderly. Additionally, the compounds of the invention are also useful for treatment of patients suffering from malaria by preventing parasite gametogenesis and fertility based on reduction of xanthurenic acid formation from its bioprecursor 3-hydroxy kynurenine.

### BACKGROUND OF THE INVENTION

1. Field of the Invention

The present invention generally relates to 4-oxo-1,4-dihydroquinoline-3-carboxylic acid compounds and their prodrug derivatives, and methods of inhibiting the enzyme kynurenine aminotransferase (KAT II) using these compounds. In particular, the invention provides 4-oxo-1,4-dihydroquinoline-3-carboxylic acid compounds and derivatives, and methods of using the same to treat neurological disorders characterized by insufficient glutamate and acetylcholine receptor function, and for the treatment of malaria.

2. Background of the Invention

Glutamate and nicotinic acetylcholine receptors are fundamentally involved in several cognitive processes. In principle, it is desirable to increase neurotransmission through these receptors to achieve physiological or clinical cognitive improvement. Studies in animals indicate that increases in glutamate and acetylcholine receptor function will prove especially beneficial in several psychiatric diseases including schizophrenia, depression, bipolar illness, attention-deficit disorder, obsessive-compulsive disorder, drug addiction, mental retardation and other neurodevelopmental disorders. Such increases will also provide substantial advantages in situations that require neuronal regeneration and synaptic plasticity.

Excitatory neurotransmission through glutamate and acetylcholine receptors can be enhanced by reducing the formation and levels of kynurenic acid, a tryptophan metabolite that normally inhibits glutamatergic and cholinergic receptor function in the brain. A major kynurenic acid-producing enzyme in the brain is kynurenine-aminotrasferase II (“KAT II”). In order to treat medical conditions that can be improved by an increase in neurotransmission through glutamate and nicotinic acetylcholine receptors, it is desirable to develop agents that inhibit KAT II, thereby reducing the formation of kynurenic acid in subjects to whom the agents are administered.

Some compounds able to reduce the formation of kynurenic acid, inter alia some benzoylalanine derivatives, have been described.

U.S. Pat. Nos. 5,519,055 and 5,708,030 to Schwarcz et al, the complete contents of which are hereby incorporated by reference, describe 5-substituted kynurenine derivatives that inhibit KAT II.

WO 2007/064784 to Schwarcz et al. describes dicarboxylic acids and derivatives or analogs thereof that inhibit KAT II.

WO 1995/003271 to Varsi et al describes 2-amino-4-phenyl-4-oxobutyric acid derivatives that inhibit kynureninase and/or kynurenine-3-hydroxylase.

There is an ongoing need to develop inhibitors of the KAT II enzyme in order to improve the treatment of disorders that can be ameliorated by inhibiting the formation of kynurenic acid.

### SUMMARY OF THE INVENTION

The present invention is based on the unexpected discovery that 4-oxo-1,4-dihydroquinoline-3-carboxylic acid derivatives are particularly effective in inhibiting the synthesis of kynurenic acid due to their ability to selectively inhibit the KAT II enzyme. The compounds are thus useful for the treatment of various neurological disorders and/or disease conditions related to glutamatergic and cholinergic receptor function. Importantly, in vivo experiments described in the Examples section below showed that administration of the compounds decreases the level of kynurenic acid produced in the mammalian brain.

The compounds of the invention have the generic formula or structure presented below (formula I)

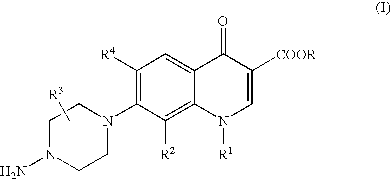

in which
R is hydrogen, C₁-C₆ alkyl or benzyl;
R¹ is C₁-C₆ alkyl, halogenated C₁-C₆ alkyl or C₁-C₆ alkylamino;
R² is hydrogen, C₁-C₆ alkyl or halogen;
or R¹ and R² may combine together to form a piperidine or a morpholine ring which is optionally substituted with a C₁-C₆ alkyl or an oxo group;
R³ is hydrogen or C₁-C₆ alkyl which is optionally substituted with halogen(s), hydroxyl(s), alkoxy(s) or amino(s) optionally substituted with alkyl(s); and
R⁴ is hydrogen, halogen or C₁-C₆ alkyl.

In addition, the invention encompasses prodrug derivatives and pharmaceutically acceptable salts of the compounds and derivatives, which may herein be referred to collectively as “compounds of the invention” or individually as “a compound of the invention”.

Further, pharmaceutical compositions that include one or more of the compounds, and/or the prodrug derivatives, and/or pharmaceutically acceptable salts of the same, are also contemplated. In such compositions, compounds of the invention are generally present in admixture with suitable excipients and/or physiologically acceptable/compatible carriers. In one embodiment of the invention, R⁴ of compound I is a halogen, for example, fluorine.

Exemplary compounds that correspond to Formula I include but are not limited to:
(S)-(−)-9-(4-Aminopiperazin-1-yl)-8-fluoro-3-methyl-6-oxo-2,3-dihydro-6H-1-oxa-3a-aza-phenalene-5-carboxylic acid,
(S)-(−)-9-(4-Aminopiperazin-1-yl)-8-fluoro-3-methyl-6-oxo-2,3-dihydro-6H-1-oxa-3a-aza-phenalene-5-carboxylic acid ethyl ester,
(R)-(+)-9-(4-Aminopiperazin-1-yl)-8-fluoro-3-methyl-6-oxo-2,3-dihydro-6H-1-oxa-3a-aza-phenalene-5-carboxylic acid,
(R)-(+)-9-(4-Aminopiperazin-1-yl)-8-fluoro-3-methyl-6-oxo-2,3-dihydro-6H-1-oxa-3a-aza-phenalene-5-carboxylic acid ethyl ester,
7-(4-Amino-3-methylpiperazin-1-yl)-6-fluoro-1-(2-fluoroethyl)-4-oxo-1,4-dihydroquinoline-3-carboxylic acid,
7-(4-Amino-3-methylpiperazin-1-yl)-1-ethyl-6-fluoro-4-oxo-1,4-dihydroquinoline-3-carboxylic acid,
8-(4-Aminopiperazin-1-yl)-9-fluoro-5-methyl-1,7-dioxo-6,7-dihydro-1H,5H-pyrido[3.2.1-ij]quinoline-2-carboxylic acid,
7-(4-Aminopiperazin-1-yl)-6-fluoro-1-(2-fluoroethyl)-4-oxo-1,4-dihydroquinoline-3-carboxylic acid,
9-(4-Aminopiperazin-1-yl)-8-fluoro-3-methyl-6-oxo-2,3-dihydro-6H-1-oxa-3a-aza-phenalene-5-carboxylic acid, and
7-(4-Aminopiperazin-1-yl)-6-fluoro-1-methylamino-4-oxo-1,4-dihydroquinoline-3-carboxylic acid. As described above, pharmaceutically acceptable salts of each of these compounds are also contemplated.

The invention also encompasses methods for inhibiting (e.g. for selectively inhibiting) the enzyme kynurenine aminotransferase. The methods include contacting the enzyme with (or alternatively, exposing the enzyme to) one or more compounds of Formula I, and/or prodrug derivatives thereof. In some embodiments, the step of contacting takes place inside cells or tissues of a mammal.

The invention also encompasses methods for treating a patient suffering from diseases characterized by a deficiency of glutamate and/or acetylcholine receptor function. The methods involve administering a compound of the invention under conditions whereby the compound inhibits the enzyme kynurenine aminotransferase. Exemplary diseases that may be treated in this manner include various neurological maladies such as schizophrenia. According to the methods, an amount of one or more compounds of the invention is administered in a quantity sufficient to inhibit kynurenine aminotransferase in the patient, and to decrease symptoms of the disease that is being treated.

Further, as described in detail below, the compounds of the invention may also be used to treat malaria.

### BRIEF DESCRIPTION OF THE DRAWINGS

FIG. 1. Measurement of kynureinc acid (KYNA) production in rat brain slices as a function of Compound (A) concentration. X axis, concentration of Compound (A) (ttL); Y axis, KYNA production as a % of control (no inhibitor).

FIG. 2. Measurement of KYNA and dopamine (DA) levels in prefrontal cortex of rat brain upon administration of Compound (A) (1 mM for 2 hours). X axis, time; Y axis, % basal levels of DA and KYNA.

### DETAILED DESCRIPTION OF THE

### Preferred Embodiments of the Invention

The present invention provides compounds of formula

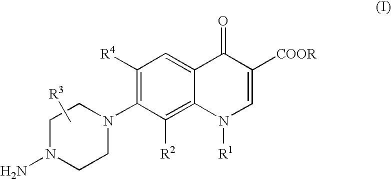

derivatives of such compounds, and methods for their use. Generally, the compounds of the invention are used to treat disease conditions that respond favourably to or which are ameliorated by inhibition of the enzyme kynurenine aminotransferase II (KAT II). Without being bound by theory, it appears that inhibition of kynurenine aminotransferase II results in a reduction in the formation and levels of kynurenic acid, a tryptophan metabolite that normally inhibits glutamatergic and cholinergic function in the brain. Conditions which are treated according to the methods of the invention may be caused by over activity of KAT II. Alternatively, the KAT II may exhibit a normal level of activity but other enzymes that act in concert with or in conjunction with KAT II (e.g. other enzymes that are active in a metabolic pathway that includes KAT II) may exhibit levels of activity that are above or below a normal level, and inhibition of KAT II may compensate for these abnormalities. Regardless of the precise mechanism of operation, any condition that is caused or exacerbated by a low level of glutamate and acetylcholine receptor function, or that can be improved by increasing glutamate and acetylcholine receptor function, may be treated by inhibiting kynurenine aminotransferase using the compounds of the invention. The compounds of the invention are thus used to indirectly increase excitatory neurotransmission through glutamate and acetylcholine receptors.

The compounds of the invention are selective for the inhibition of KAT II. In other words, the inhibition of KAT II by the compounds of the invention, when compared to inhibition of other physiologically relevant enzymes (i.e. enzymes which are also likely to be exposed to the compound when it is administered to an individual) is at least 2-fold greater, preferably 5, 10, 20, 30, 40 50, 60, 70, 80, 90 or 100-fold, or even 200, 300, 400, 500, 600, 700, 800, 900, or 1000-fold or more, greater. Typically, the level of inhibition of an enzyme is determined by measuring the IC₅₀ of the enzyme for the compound, as will be readily understood by those of skill in the art.

In this specification, the term “derivative” means a prodrug of the compound (I). The prodrug refers to a compound (e.g. a drug precursor) that is transformed in vivo to yield the compound (I). The transformation may occur by various mechanisms (e.g., by metabolic or chemical processes), such as, for example, through hydrolysis in blood. Examples of prodrugs and their use are described in T. Higuchi and W. Stella, “Pro-drugs as Novel Delivery Systems”, Vol. 14 of the A.C.S. Symposium Series; Edward B. Roche, “Bioreversible Carriers in Drug Design”, American Pharmaceutical Association and Pergamon Press, 1987; “Development of Drugs”, Vol. 7, Molecular Design, Hosokawa Shoten, 1990, p. 163-198; “Prog Med”, 1985, Vol. 5, p. 2157-2161; D. Fleisher, S. Ramon and H. Barbra “Improved oral drug delivery: solubility limitations overcome by the use of prodrugs”, Advanced Drug Delivery Reviews, 1996, Vol. 19(2), p. 115-130.

For example, if a compound (I) contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

A prodrug can be also formed by the replacement of a hydrogen atom in the amine group of compound (I) with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

The present invention also includes isomers, including optical isomers, enantiomers, stereoisomers, diastereomers, or racemic mixtures of compounds corresponding to formula (I).

In this specification, C₁-C₆ alkyl includes but is not limited to, for example, methyl, ethyl, propyl, isopropyl, butyl, isobutyl, sec-butyl, tert-butyl, penty, isopentyl, hexyl, etc. As the skilled artisan is aware and recognizes, the C₁-C₆ alkyl may be straight chain or branched, such as isopropyl or tert-butyl alkyl substituents. Halogenated C₁-C₆ alkyl includes but is not limited to, for example, fluoromethyl, difluoromethyl, trifluoromethyl, 2-fluoroethyl, etc. C₁-C₆ Alkylamino includes but is not limited to, for example, methylamino, ethylamino, dimethylamino, diethylamino. Halogen includes chlorine, fluorine, iodine, bromine, etc.

The pharmaceutically acceptable salts contemplated include but are not limited to a salt of HCl, HBr, HNO₃, H₂SO₄, acetic acid, maleic acid, succinic acid, and trifluoroacetic acid, etc.

The compounds of formula (I) can be prepared according to the following methods:

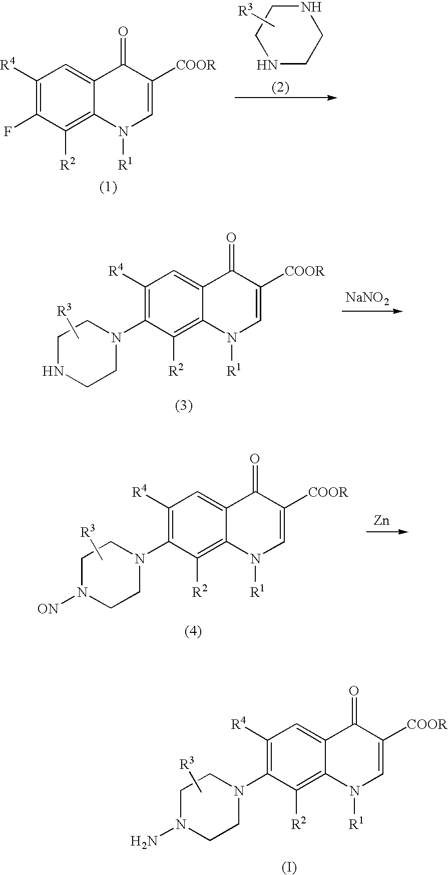

The quinolones, such as (3) are readily prepared from a compound having a fluoroquinolone core, such as (1), and a nucleophilic amine, such as (2), according to the procedures described in the literature such as, McGuirk, P. R. et al: J. Med. Chem. 1992, 35, 611-620. Hydrazine formation can be achieved using a two-step procedure, which is nitrosylation of the amine in (3) with a nitrosylating agent, like sodium nitrite in aqueous acidic solution, followed by reduction with zinc to yield compound (I), having a hydrazine in the A-ring.

The compounds of formula (I) inhibited kynurenine aminotransferase activity with IC₅₀ values ranging from 0.2 to 2 μM, and proved particularly selective for this enzyme (significant inhibition of kynurenine 3-hydroxylase and kynureninase, other enzymes involved in the kynurenine pathway metabolism, is only observed at values above 1000 μM). Therefore, the compounds of the invention can be used for the preparation of pharmaceutical compositions for the treatment of psychiatric, neurological, and neurodegenerative diseases, in particular schizophrenia, depression, bipolar illness, attention-deficit disorder, obsessive-compulsive disorder, anxiety, drug addiction, mental retardation, Parkinson's disease, Alzheimer's disease, cognitive disorders associated with neurodegenerative and seizure disorders, age-related cognitive deficit, cognitive disorders in children, as well as for the stimulation of neuronal regeneration in neurodegenerative and seizure disorders, and after cell transplantation. Other conditions which may be treated by administration of the compounds of the invention include but are not limited to multiple sclerosis, amyotrophic lateral sclerosis, spinal muscular atrophy, peripheral neuropathy, Creutzfeldt-Jakob disease, AIDS dementia, progressive supranuclear palsy, myelinopathia centralis diffusa (vanishing white matter disease), chronic neurodegenerative disease, Huntington's disease, optic neuropathy, optic neuritis, Down's syndrome, encephalomyelitis, meningitis, panencephalitis, lewy body dementia, myasthenia gravis, congenital ornithine transcarbamylase (OTC) deficiency, glutaryl-CoA dehydrogenase (GCDH) deficiency, and narcolepsy.

The malaria parasite Plasmodium falciparum is carried by Anopheles gambiae, and xanthurenic acid plays a key role in parasite gametogenesis and fertility (Vernick K D. Cell 117, 419-420, 2004). KAT II is also known to produce xanthurenic acid from its bioprecursor 3-hydroxykynurenine (Takeuchi F et al. J Nutr Sci Vitaminol (Tokyo) 35, 111-122, 1989). Therefore, the compounds of the invention are also useful for the treatment of patients suffering from malaria, since administration of the compounds to patients suffering from, or likely to contract, malaria would inhibit the production of xanthurenic acid. Hence, the reproduction of the parasite would be prevented.

The pharmaceutical compositions (formulations) can be prepared according to methods commonly known to those skilled in the art, in particular according to what described in Remington's Pharmaceutical Sciences Handbook, XVII Ed. Mack Pub., N.Y., U.S.A. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active agent in the formulations may vary. However, in general, the amount in the formulations will be from about 1-99%.

The compositions (preparations) of the present invention may be administered by any of the many suitable means which are well known to those of skill in the art, including but not limited to by injection, orally, etc. In preferred embodiments, the mode of administration is oral preparations such as tablets, capsules, powders, fine granules, granules, solutions and syrups, and parenteral preparations such as injections. Administration may be systemic, or, in some cases, may be directed to a particular organ or tissue type, e.g. to the brain. In addition, the compositions may be administered in conjunction with other treatment modalities such as other psychotropic agents, etc. Further, in some embodiments only one form of the compounds are administered, but this need not be the case. For example, a mixture of two or more of the compounds may be present in a single composition, or may be administered together in separate compositions. This may be especially advantageous, for example, if a combination of long acting and immediately active forms is used. For example, by using a prodrug form of a compound (e.g. a compound that is inactive or has low activity prior to conversion, within the body, to an active form) in combination with a form of a compound that is immediately active upon administration, a desirable long-acting effect may be achieved. This could be especially helpful for patients with psychological impairments, who may, for example, have difficulty keeping track of medications, in that the medication would need to be taken or administered less frequently

As used herein, the terms “effective amount” or “therapeutically effective amount” are interchangeable and refer to an amount that results in an improvement or remediation of at least one symptom of the disease or condition. Those of skill in the art understand that the effective amount may improve the patient's or subject's condition, but may or may not result in a complete cure of the disease and/or condition.

An effective amount of a therapeutic composition of the invention, including an inhibitor of KAT II and/or the additional therapeutic compounds that may be administered to a cell includes a dose of about 0.0001 nM to about 2000 μM, for example. More specifically, doses to be administered are from about 0.001 μM to about 0.01 μM; about 0.01 nM to about 2000 μM; about 0.01 μM to about 0.05 μM; about 0.05 μM to about 1.0 μM; about 1.0 μM to about 1.5 μM; about 1.5 μM to about 2.0 μM; about 2.0 μM to about 3.0 μM; about 3.0 μM to about 4.0 μM; about 4.0 μM to about 5.0 μM; about 5.0 μM to about 10 μM; about 10 μM to about 50 μM; about 50 μM to about 100 μM; about 100 μM to about 200 μM; about 200 μM to about 300 μM; about 300 μM to about 500 μM; about 500 μM to about 1000 μM; about 1000 μM to about 1500 μM and about 1500 μM to about 2000 μM, for example. Of course, all of these amounts are exemplary, and any amount in-between these points is also expected to be of use in the invention.

An effective amount of an inhibitor of KAT II such as those described herein as a treatment varies depending upon the host treated and the particular mode of administration. In one embodiment of the invention, the dose range of the therapeutic combinatorial composition of the invention, including an inhibitor of KAT II and/or the additional therapeutic compound, will be about 0.01 μg/kg body weight to about 20,000 μg/kg body weight. The term “body weight” is applicable when an animal is being treated. When isolated cells are being treated, “body weight” as used herein should read to mean “total cell body weight”. The term “total body weight” may be used to apply to both isolated cell and animal treatment. All concentrations and treatment levels are expressed as “body weight” or simply “kg” in this application are also considered to cover the analogous “total cell body weight” and “total body weight” concentrations. However, those of skill will recognize the utility of a variety of dosage range, for example, 0.01 μg/kg body weight to 20,000 μg/kg body weight, 0.02 μg/kg body weight to 15,000 μg/kg body weight, 0.03 μg/kg body weight to 10,000 μg/kg body weight, 0.04 μg/kg body weight to 5,000 μg/kg body weight, 0.05 μg/kg body weight to 2,500 μg/kg body weight, 0.06 μg/kg body weight to 1,000 μg/kg body weight, 0.07 μg/kg body weight to 500 μg/kg body weight, 0.08 μg/kg body weight to 400 μg/kg body weight, 0.09 μg/kg body weight to 200 μg/kg body weight or 0.1 μg/kg body weight to 100 μg/kg body weight. Further, those of skill will recognize that a variety of different dosage levels will be of use, for example, 0.0001 μg/kg, 0.0002 μg/kg, 0.0003 μg/kg, 0.0004 μg/kg, 0.005 μg/kg, 0.0007 μg/kg, 0.001 μg/kg, 0.1 μg/kg, 1.0 μg/kg, 1.5 μg/kg, 2.0 μg/kg, 5.0 μg/kg, 10.0 μg/kg, 15.0 μg/kg, 30.0 μg/kg, 50 μg/kg, 75 μg/kg, 80 μg/kg, 90 μg/kg, 100 μg/kg, 120 μg/kg, 140 μg/kg, 150 μg/kg, 160 μg/kg, 180 μg/kg, 200 μg/kg, 225 μg/kg, 250 μg/kg, 275 μg/kg, 300 μg/kg, 325 μg/kg, 350 μg/kg, 375 μg/kg, 400 μg/kg, 450 μg/kg, 500 μg/kg, 550 μg/kg, 600 μg/kg, 700 μg/kg, 750 μg/kg, 800 μg/kg, 900 μg/kg, 1 mg/kg, 5 mg/kg, 10 mg/kg, 12 mg/kg, 15 mg/kg, 20 mg/kg, and/or 30 mg/kg.

In particular embodiments, there may be dosing of from very low ranges (e.g. 1 mg/kg/day or less; 5 mg/kg bolus; or 1 mg/kg/day) to moderate doses (e.g. 2 mg bolus, 15 mg/day) to high doses (e.g. 5 mg bolus, 30-40 mg/day; and even higher). Of course, all of these dosages are exemplary, and any dosage in-between these points is also expected to be of use in the invention.

In certain embodiments, the amount of the combinatorial therapeutic composition administered to the subject is in the range of about 0.0001 μg/kg/day to about 250 μg/kg/day, about 0.01 μg/kg/day to about 100 μg/kg/day, or about 1 μg/kg/day to about 50 μg/kg/day, or about 5 μg/kg/day to about 20 μg/kg/day. Still further, the combinatorial therapeutic composition may be administered to the subject in the form of a treatment in which the treatment may comprise the amount of the combinatorial therapeutic composition or the dose of the combinatorial therapeutic composition that is administered per day (1, 2, 3, 4, etc.), week (1, 2, 3, 4, 5, etc.), month (1, 2, 3, 4, 5, etc.), etc. Treatments may be administered such that the amount of combinatorial therapeutic composition administered to the subject is in the range of about 0.0001 μg/kg/treatment to about 1 mg/kg/treatment, about 0.01 μg/kg/treatment to about 100 μg/kg/treatment, or about 1 μg/kg/treatment to about 10 μg/kg/treatment.

As used herein, the term “inhibit” refers to the ability of the compound to block, partially block, interfere, decrease, reduce or deactivate an enzyme such as kynurenine aminotransferase (KAT II). Thus, one of skill in the art understands that the term inhibit encompasses a complete and/or partial loss of activity of an enzyme such as KAT II. Enzymatic activity may be inhibited by a block (occlusion of the active site), by changes in the activity, or by other means. For example, a complete and/or partial loss of activity of the KAT II may be indicated by a reduction in kynurenic acid levels in body fluids, tissue such as brain and peripheral tissue, blood, serum or the like. Generally, the level of inhibition will be in the range of at least from about 25%, 30%, 35%, 40%, 45%, 50%, 55%, 60%, 65%, 70%, 75%, 80%, 85%, 90%, or 95% to 100%, as measured under standardized conditions using methodology that is recognized by those of skill in the art, e.g. the assays that are employed in the Examples section below.

In the invention also provides a kit for the inhibition of the KAT II enzyme and/or for the treatment of a condition amenable to treatment by inhibiting KAT II. The kit comprises an inhibitor of KAT II housed in a suitable container. The kit may also comprise suitable tools to administer compositions of the invention to an individual.

The invention is further illustrated by the following examples.

### EXAMPLES

### Example 1

### Synthesis of (S)-(−)-9-(4-Aminopiperazin-1-yl)-8-fluoro-3-methyl-6-oxo-2,3-dihydro-6H-1-oxa-3a-aza-phenalene-5-carboxylic acid trifluoroacetate

An exemplary compound of the invention, (S)-(−)-9-(4-Aminopiperazin-1-yl)-8-fluoro-3-methyl-6-oxo-2,3-dihydro-6H-1-oxa-3a-aza-phenalene-5-carboxylic acid trifluoroacetate, (hereinafter this compound is called as “Compound (A)”, was synthesized according to the following scheme:

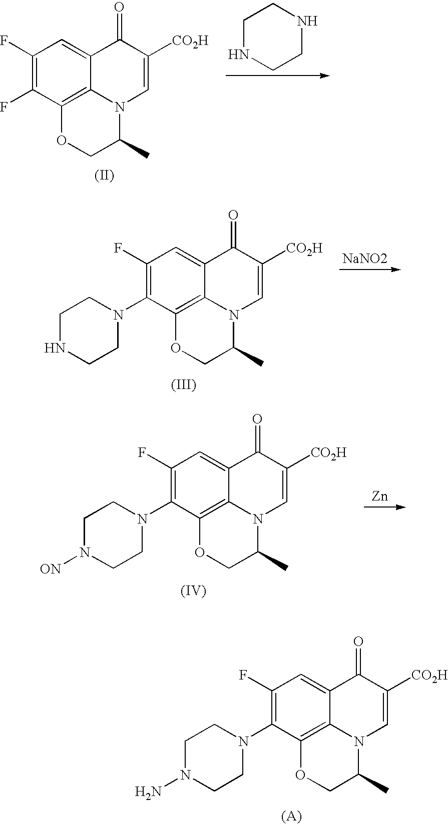

A solution of (S)-(−)-9,10-Difluoro-2,3-dihydro-3-methyl-7-oxo-7H-pyrido[1,2,3-de][1,4]benzoxazine-6-carboxylic acid (II) (ALDRICH, 10.0 g, 35.3 mmol) and piperazine (24.3 g, 283 mmol) in dimethylsulfoxide (DMSO) (100 ml) was stirred for 8 h at 100 deg. The reaction mixture was cooled to room temperature and concentrated in vacuo. The residue was diluted with 97% methanol and yielded a compound of formula (III) 9.34 g (76%) as a yellow solid.

NaNO₂ (5.37 g, 77.8 mmol)-H₂O (40 ml) solution was added portion to a solution of compound (III) (5.40 g, 15.5 mmol) in acetic acid (70 ml)-H₂O (70 ml), and the reaction mixture was stirred for 4.5 h at room temperature. The mixture was filtered, washed with water, dried and yielded ca. 13 g of compound (IV). This compound was used without purification.

Activated Zn (5.85 g) was added to a solution of compound (IV) (5.83 g, 15.5 mmol) in acetic acid (120 ml)-H₂O (120 ml). The reaction mixture was heated at 70° C. for 45 min, then cooled to room temperature and concentrated in vacuo. The residue was purified by HPLC (ODS, H₂O/MeCN/TFA) to yield Compound (A) as a white solid (trifluoroacetate salt) with the following characteristics:

FAB-MS: 363 [MH⁺]

¹H-NMR (DMSO-d6):

8.98(s, 1H), 7.61 (d, J=12.0 Hz, 1H), 4.97-4.91 (m, 1H), 4.59 (dd, J=10.8, 0.8 Hz, 1H), 4.38 (dd, J=11.6, 2.4 Hz), 3.50-3.40 (m, 4H), 3.10-3.00 (m, 4H), 1.45 (d, J=6.8 Hz, 3H).

### Example 2

### 7-(4-Amino-3-methylpiperazin-1-yl)-6-fluoro-1-(2-fluoroethyl)-4-oxo-1,4-dihydroquinoline-3-carboxylic acid

7-(4-Amino-3-methylpiperazin-1-yl)-6-fluoro-1-(2-fluoroethyl)-4-oxo-1,4-dihydroquinoline-3-carboxylic acid was prepared essentially as described in Example 1.

The resulting compound displayed the following characteristics:

ESI-MS m/z: 367 [M+H⁺]

¹H-NMR (DMSO-d6)

9.65(br s, 2H), 8.92 (s, 1H), 7.97 (d, J=13.1 Hz, 1H), 7.25 (m, 1H), 4.78-5.02 (m, 4H), 2.87-3.72 (m, 7H), 1.19 (s, 3H).

### Example 3

### 7-(4-Amino-3-methylpiperazin-1-yl)-1-ethyl-6-fluoro-4-oxo-1,4-dihydroquinoline-3-carboxylic acid

7-(4-Amino-3-methylpiperazin-1-yl)-1-ethyl-6-fluoro-4-oxo-1,4-dihydroquinoline-3-carboxylic acid is prepared in a manner similar to that of Example 1.

### Example 4

### 8-(4-Aminopiperazin-1-yl)-9-fluoro-5-methyl-1,7-dioxo-6,7-dihydro-1H,5H-pyrido[3.2.1-ij]quinoline-2-carboxylic acid

8-(4-Aminopiperazin-1-yl)-9-fluoro-5-methyl-1,7-dioxo-6,7-dihydro-1H,5H-pyrido[3.2.1-ij]quinoline-2-carboxylic acid is prepared in a manner similar to that of Example 1.

### Example 5

### 7-(4-Aminopiperazin-1-yl)-6-fluoro-1-(2-fluoroethyl)-4-oxo-1,4-dihydroquinoline-3-carboxylic acid

7-(4-Aminopiperazin-1-yl)-6-fluoro-1-(2-fluoroethyl)-4-oxo-1,4-dihydroquinoline-3-carboxylic acid was prepared essentially as described in Example 1.

The resulting compound displayed the following characteristics:

ESI-MS m/z: 353 [M+H⁺]

¹H-NMR (DMSO-d6)

8.89(s, 1H), 7.93 (d, J=13.4 Hz, 1H), 7.20 (d, J=7.2 Hz, 1H), 4.78-5.01 (m, 4H), 3.34 (m, 4H), 2.70 (m, 4H).

### Example 6

### 9-(4-Aminopiperazin-1-yl)-8-fluoro-3-methyl-6-oxo-2,3-dihydro-6H-1-oxa-3a-aza-phenalene-5-carboxylic acid

9-(4-Aminopiperazin-1-yl)-8-fluoro-3-methyl-6-oxo-2,3-dihydro-6H-1-oxa-3a-aza-phenalene-5-carboxylic acid was prepared essentially as described in Example 1.

The resulting compound displayed the following characteristics:

ESI-MS m/z: 363 [M+H⁺]

¹H-NMR (DMSO-d6) 8.98(s, 1H), 7.61 (d, J=12.0 Hz, 1H), 4.97-4.91 (m, 1H), 4.59 (dd, J=10.8, 0.8 Hz, 1H), 4.38 (dd, J=11.6, 2.4 Hz), 3.50-3.40 (m, 4H), 3.10-3.00 (m, 4H), 1.45 (d, J=6.8 Hz, 3H).

### Example 7

### 7-(4-Aminopiperazin-1-yl)-6-fluoro-1-methylamino-4-oxo-1,4-dihydroquinoline-3-carboxylic acid

7-(4-Aminopiperazin-1-yl)-6-fluoro-1-methylamino-4-oxo-1,4-dihydroquinoline-3-carboxylic acid was prepared essentially as described in Example 1.

The resulting compound displayed the following characteristics:

ESI-MS m/z: 336 [M+H⁺]

¹H-NMR (DMSO-d6) 9.79(br s, 2H), 8.94 (s, 1H), 7.92 (d, J=13.1 Hz, 1H), 7.70 (d, J=7.7 Hz, 1H), 7.31(br s, 1H), 3.40 (m, 4H), 3.15 (m, 4H), 2.85 (s, 3H).

### Example 8

### (R)-(+)-9-(4-Aminopiperazin-1-yl)-8-fluoro-3-methyl-6-oxo-2,3 dihydro-6H-1-oxa-3a-aza-phenalene-5-carboxylic acid trifluoroacetate

(R)-(+)-9-(4-Aminopiperazin-1-yl)-8-fluoro-3-methyl-6-oxo-2,3-dihydro-6H-1-oxa-3a-aza-phenalene-5-carboxylic acid trifluoroacetate was prepared essentially as described in Example 1, except that (R)-(+)-9,10-Difluoro-2,3-dihydro-3-methyl-7-oxo-7H-pyrido[1,2,3-de][1,4]benzoxazine-6-carboxylic acid was used instead of (S)-(−)-9,10-Difluoro-2,3-dihydro-3-methyl-7-oxo-7H-pyrido[1,2,3-de][1,4]benzoxazine-6-carboxylic acid (II).

The resulting compound displayed the following characteristics:

ESI-MS m/z 363 [M+H⁺]

¹H-NMR (DMSO-d6)

9.38(br s, 2H), 9.00 (s, 1H), 7.61 (d, J=12.4 Hz, 1H), 4.97-4.91 (m, 1H), 4.59 (dd, J=10.8, 0.8 Hz, 1H), 4.38 (dd, J=11.6, 2.4 Hz), 3.50-3.40 (m, 4H), 3.10-3.00 (m, 4H), 1.45 (d, J=6.8 Hz, 3H).

### Example 9

### (S)-(−)-9-(4-Aminopiperazin-1-yl)-8-fluoro-3-methyl-6-oxo-2,3-dihydro-6H-1-oxa-3a-aza-phenalene-5-carboxylic acid ethyl ester

(S)-(−)-9-(4-Aminopiperazin-1-yl)-8-fluoro-3-methyl-6-oxo-2,3-dihydro-6H-1-oxa-3a-aza-phenalene-5-carboxylic acid ethyl ester was prepared essentially as described in Example 1.

The resulting compound displayed the following characteristics:

¹H-NMR (DMSO-d6)

9.05(br s, 2H), 8.63 (s, 1H), 7.46 (d, J=12.2 Hz, 1H), 4.75 (m, 1H), 4.53 (dd, J=11.3, 1.5 Hz, 1H), 4.34 (dd, J=11.3, 2.2 Hz, 1H), 4.22 (m, 2H), 3.43 (m, 4H), 3.12 (m, 4H), 1.41 (d, J=6.7 Hz, 3H), 1.28 (m, 3H).

### Example 10

### In Vitro Testing of KAT Inhibitors

For the measurement of KAT II activity, 100 μl of partially purified rat liver enzyme were incubated (2 hrs, 37° C.) with 100 μl of 150 mM Tris-acetate (pH 7.0), 2 μM (2.5 nCi) [³H]-kynurenine, 1 mM pyruvate and 80 μM pyridoxal-5′-phosphate. Blanks were obtained using denatured protein preparations. The reaction was terminated by the addition of 25 μM of 30% (w/v) trichloroacetic acid. 1 ml of 0.1 M HCl was added, and the denatured protein was removed by centrifugation. 1 ml of the supernatant was applied to a Dowex 50 W H⁺cation exchange column, which was then washed with 1 ml of 0.1 M HCl, followed by 1 ml of ultrapure water. [³H]-KYNA was subsequently eluted with 2×1 ml of ultrapure water, and radioactivity was quantified by liquid scintillation spectrometry. Human recombinant KAT II protein was also used to measure KAT II activity as described above.

Test compounds were added in 20 μl aliquots at the beginning of the incubation period to examine interference with enzyme activity.

Kynurenine 3-hydroxylase and kynureninase activities were determined in rat liver homogenate according to established procedures (see Anal. Biochem., 205:257-62, 1992 and Neuroscience., 61:237-43, 1994 for methodology).

Microdialysis was performed in the prefrontal cortex of unanesthetized male rats (200-220 g) according to established procedures (see Eur. J. Neurosci., 4:1264-70, 1992 for methodology).

More particularly, a comparative analysis of Compound (A) and known KAT inhibitor UPF 874 (compound (B), which is described in detail in U.S. Pat. No. 5,688,945, the complete contents of which is whereby incorporated by reference) yielded the following results (IC₅₀ values are expressed in μM):

Compound (A)

KAT II: 0.2 (human recombinant protein) or 2 (partially purified rat enzyme)

Kynurenine 3-hydroxylase: >1000

Kynureninase: >2000

Compound (B)

KAT II: 1000 (human recombinant protein) or 6 (partially purified rat enzyme)

Kynurenine 3-hydroxylase: >1000

Kynureninase: >2000

As demonstrated herein, UPF 874 is almost 200 times less active against human KAT II than against rat KAT II or, put differently, Compound (A) is 10 times more active against human KAT II than against rat KAT II. Therefore, it is concluded that Compound (A) is 3000 times more active than UPF 874 as a KAT II inhibitor in humans and provides a novel class of compounds having the ability to treat diseases associated with aberrant kynurenic acid formation or function.

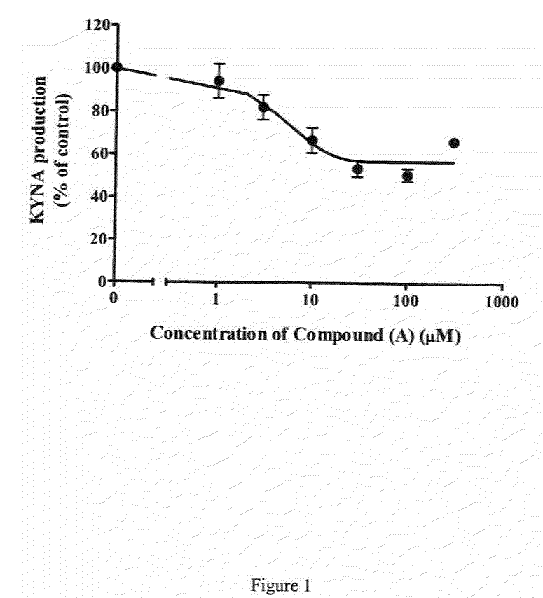

The ability of Compound (A) to inhibit the de novo production of KYNA in rat brain tissue slices was also tested. A 2 μM concentration of Compound (A) was assayed as described by Gramsbergen J B, Hodgkins P S, Rassoulpour A, Turski W A, Guidetti P, Schwarcz R., (J. Neurochem., 69:290, 1997). Briefly, an aliquot of the original tissue homogenate was further diluted (1:5, v/v) in a buffer containing 5 mM Tris acetate (pH 8.0), 50 μM pyridoxal-5′-phosphate and 10 mM 2-mercapto-ethanol, and dialyzed overnight at 4 degrees. The reaction mixture containing 150 mM Tris-acetate buffer (pH 7.0), 2 μM L-kynurenine, 1 mM pyruvate, 80 μM pyridoxal-5′-phosphate and 80 μL dialysate in a total volume of 200 μM was incubated w/wo Compound (A) for 6 hr at 37 degrees. The reaction was terminated by adding 10 μA of 50% (w/v) trichloracetic acid and KYNA levels were measured using reverse-phase HPLC with fluorescence detection (excitation, 344 nm; emission, 398 nm). The results are presented in FIG. 1. As can be seen, Compound (A) was found to dose-dependently inhibit the de novo production of KYNA from its bioprecursor L-kynurenine.

### Example 11

### In vivo Testing of KAT II Inhibitors

Compound (A) was also tested in the rat brain in vivo. Briefly, a concentration of 1 mM Compound (A) was introduced into the prefrontal cortex for 2 hours by reverse dialysis. A detailed description of this method can be found in J Neurosci Res 85(4):845-54, 2007 (Ceresoli-Borroni G, Guidetti P, Amori L, Pellicciari R, Schwarcz R).

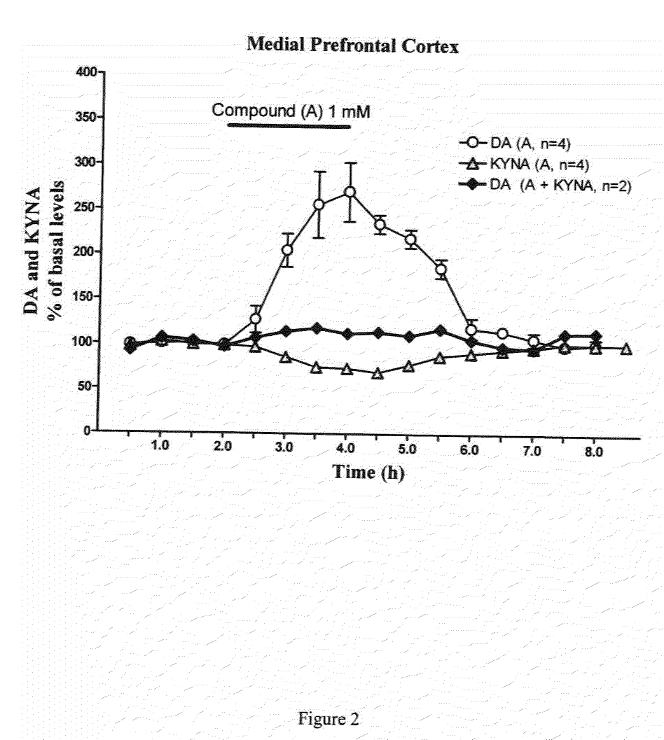

The results are presented in FIG. 2. As can be seen, Compound (A) was reduced the extracellular concentration of kynurenic acid and, at the same time, induced the extracellular concentration of dopamine in the prefrontal cortex. Specifically, administration of Compound (A) reversibly reduced extracellular kynurenic acid (KYNA) levels by ˜40% and simultaneously increased extracellular dopamine (DA) levels >2.5-fold. To ascertain the direct causal relationship between the reduction of KYNA and the DA increase, separate rats were perfused with 1 mM Compound (A) and 100 nM KYNA (n=2). In quantitative terms, the reduction of kynurenic acid and increase of dopamine were similar to the results observed after the in vivo administration of known cognition-enhancing drugs such as d-amphetamine and methylphenidate. Such decreases in hippocampalkynurenic acid levels are known to enhance the activity of u7 nicotinic acetylcholine receptors (Alkondon et al., J. Neurosci., 24: 4635-4648, 2004).

The results obtained with exemplary Compound (A) thus show that the compounds of the invention can be used to increase cholinergic function in vivo, for the treatment of cognitive deficits as described herein.

The foregoing has outlined rather broadly the features and technical advantages of the present invention in order that the detailed description of the invention that follows may be better understood. Additional features and advantages of the invention will be described herein which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features, which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

We claim:

1. A compound of formula (I) 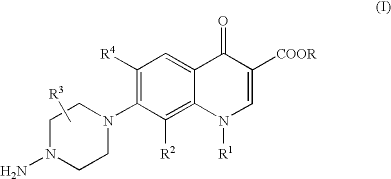
in which
R is hydrogen, C₁-C₆ alkyl or benzyl;
R¹ is C₁-C₆ alkyl, halogenated C₁-C₆ alkyl or C₁-C₆ alkylamino;
R² is hydrogen, C₁-C₆ alkyl or halogen;
or R¹ and R² may combine together to form a piperidine or a morpholine ring which is optionally substituted by a C₁-C₆ alkyl or an oxo group;
R³ is hydrogen or C₁-C₆ alkyl which is optionally substituted with halogen(s), hydroxyl(s), alkoxy(s) or amino(s) optionally substituted with alkyl(s); and
R⁴ is hydrogen, halogen or C₁-C₆ alkyl; or
its derivative or a pharmaceutically acceptable salt thereof.

2. A compound as claimed in claim 1 wherein R⁴ is halogen.

3. A compound as claimed in claim 1 wherein R⁴ is fluorine.

4. A compound as claimed in claim 1, wherein the compound is (S)-(−)-9-(4-Aminopiperazin-1-yl)-8-fluoro-3-methyl-6-oxo-2,3-dihydro-6H-1-oxa-3a-aza-phenalene-5-carboxylic acid or a pharmaceutically acceptable salt thereof.

5. A compound as claimed in claim 1, wherein the compound is (5)-(−)-9-(4-Aminopiperazin-1-yl)-8-fluoro-3-methyl-6-oxo-2,3-dihydro-6H-1-oxa-3a-aza-phenalene-5-carboxylic acid ethyl ester or a pharmaceutically acceptable salt thereof.

6. A compound as claimed in claim 1 wherein the compound is (R)-(+)-9-(4-Aminopiperazin-1-yl)-8-fluoro-3-methyl-6-oxo-2,3-dihydro-6H-1-oxa-3a-aza-phenalene-5-carboxylic acid or a pharmaceutically acceptable salt thereof.

7. A compound as claimed in claim 1 wherein the compound is (R)-(+)-9-(4-Aminopiperazin-1-yl)-8-fluoro-3-methyl-6-oxo-2,3-dihydro-6H-1-oxa-3a-aza-phenalene-5-carboxylic acid ethyl ester or a pharmaceutically acceptable salt thereof.

8. A compound as claimed in claim 1 wherein the compound is 7-(4-Amino-3-methylpiperazin-1-yl)-6-fluoro-1-(2-fluoroethyl)-4-oxo-1,4-dihydroquinoline-3-carboxylic acid or a pharmaceutically acceptable salt thereof.

9. A compound as claimed in claim 1 wherein the compound is 7-(4-Amino-3-methylpiperazin-1-yl)-1-ethyl-6-fluoro-4-oxo-1,4-dihydroquinoline-3-carboxylic acid or a pharmaceutically acceptable salt thereof.

10. A compound as claimed in claim 1 wherein the compound is 8-(4-Aminopiperazin-1-yl)-9-fluoro-5-methyl-1,7-dioxo-6,7-dihydro-1H,5H-pyrido[3.2.1-ij]quinoline-2-carboxylic acid or a pharmaceutically acceptable salt thereof.

11. A compound as claimed in claim 1 wherein the compound is 7-(4-Aminopiperazin-1-yl)-6-fluoro-1-(2-fluoroethyl)-4-oxo-1,4-dihydroquinoline-3-carboxylic acid or a pharmaceutically acceptable salt thereof.

12. A compound as claimed in claim 1 wherein the compound is 9-(4-Aminopiperazin-1-yl)-8-fluoro-3-methyl-6-oxo-2,3-dihydro-6H-1-oxa-3a-aza-phenalene-5-carboxylic acid or a pharmaceutically acceptable salt thereof.

13. A compound as claimed in claim 1 wherein the compound is 7-(4-Aminopiperazin-1-yl)-6-fluoro-1-methylamino-4-oxo-1,4-dihydroquinoline-3-carboxylic acid or a pharmaceutically acceptable salt thereof.

14. A method for inhibiting kynurenine aminotransferase (KAT II), which comprises administering a compound of claim 1, wherein said compound selectively inhibits KAT II.

15. A method for treating a disease associated with aberrant kynurenic acid levels in vivo comprising administering to a subject in need thereof an effective amount of a compound of claim 1.

16. The method of claim 15, wherein the disease is a neurodegenerative disease.

17. The method of claim 15, wherein the disease is selected from the group consisting of malaria, schizophrenia, depression, bipolar illness, attention-deficit disorder, obsessive-compulsive disorder, drug addiction, mental retardation, Parkinson's disease, Alzheimer's disease, cognitive disorders in neurodegenerative and seizure disorders, age-related cognitive deficit, or cognitive disorders in children.

18. The method of claim 15, wherein the compound is (S)-(−)-9-(4-Aminopiperazin-1-yl)-8-fluoro-3-methyl-6-oxo-2,3-dihydro-6H-1-oxa-3a-aza-phenalene-5-carboxylic acid or a pharmaceutically acceptable salt thereof.

19. The method of claim 15, wherein the disease is Alzheimer's disease.

20. The method of claim 15, wherein the disease is schizophrenia.
